(19) United States
(12) Patent Application Publication
(10) Pub. No.: US 2020/0338359 A1
(43) Pub. Date: Oct. 29, 2020

(54) EXTRACTION OF STEM CELLS FROM BONE MARROW NICHES

(71) Applicant: Roemer & Heigl GmbH, Basel (CH)

(72) Inventors: Ramona ROEMER, Basel (CH); Johannes Heinrich ESS, Daleiden (DE)

(73) Assignee: Roemer & Heigl GmbH, Basel (CH)

(21) Appl. No.: 16/760,733

(22) PCT Filed: Nov. 2, 2018
(86) PCT No.: PCT/EP2018/079988

§ 371 Date: Apr. 30, 2020

(30) Foreign Application Priority Data
Nov. 3, 2017 (EP) 17199938.6

(51) Int. Cl. A61N 5/06; A61K 35/28

(52) U.S. Cl. CPC A61N 5/0603; A61K 9/0019; A61N 2005/0602; A61K 35/28

(57) ABSTRACT

The subject-matter of the present invention is a method for reversibly releasing stem cells from bone marrow niches into peripheral blood and isolating these stem cells. Further, the present invention relates to stem cells obtained in this manner and their use in the medical treatment.

The subject-matter of the present invention is a method for reversibly releasing stem cells from bone marrow niches into peripheral blood and isolating these stem cells. Further, the present invention relates to stem cells obtained in this manner and their use in the medical treatment.

Hematopoietic stem cells are the stem cells that give rise to all other blood cells through the process of hematopoiesis. They are found in the bone marrow, especially in the pelvis, femur and sternum and in small amounts also in peripheral blood. In order to extract hematopoietic stem cells for medical purposes they can be harvested from bone marrow. As an alternative technique, hematopoietic stem cells are commonly obtained from peripheral blood through a process known as apheresis. Since the number of stem cells in the blood is normally too little to obtain larger amounts of them, it is necessary to mobilise stem cells from their site of origin into circulation and increase their number in peripheral blood, thus allowing a more efficient collection of an increased amount of stem cells from the blood circulation. This can be done, for instance, with cytokines such as granulocyte-colony stimulating factor (G-CSF) and granulocyte-macrophage colony-stimulating factor (GM-CSF) that induce cells to leave the bone marrow and circulate in the blood vessels. Other examples of factors and agents that are capable of mobilizing stem cells into circulating blood of a subject are CXCR4-receptor inhibitors such as Mozobil™.

International application WO 2008/019371 describes a combination of G-CSF with at least one CXCR4 inhibitor and at least one CXCR2 agonist for mobilizing stem cells into the bloodstream of a subject. U.S. Pat. No. 6,875,753 describes the administration of hyaluronic acid having a molecular weight of less than about 750,000 Da to a stem cell donor for increasing the concentration of blood stem cells in the blood of the donor. WO 2011/138512 describes the combined use of at least one sulphated hyaluronan oligomer or polymer and at least one factor capable of releasing stem cells such as G-CSF. In US application 2015/0374736 uridine diphosphate-glucose is used for mobilizing hematopoietic stem cells from the bone marrow into the peripheral circulation of a subject.

The disadvantage of the methods for mobilizing hematopoietic stem cells known so far in the prior art is, however, that the release of the stem cells from their anchoring in bone marrow niches is irreversible. This means that the stem cells cannot return to their natural parking place in the bone marrow niche once they have entered the circulating peripheral blood. They are lost to the body after the above or similar medicaments have been applied. Moreover, adverse undesired effects related to the administration of factors capable of mobilizing stem cells (e.g. G-CSF) such as reduced immunodefense/immunosuppression are unfavourable for a patient and hence should be avoided.

Thus, there was a need for new, improved methods that enable the mobilization of stem cells from their place of origin and increase them in the bloodstream without affecting a stem cell's ability to anchor in bone marrow niches. The reversible detachment of stem cells would be a preferable alternative to the irreversible detachment applied so far.

In the present invention, it was surprisingly found that stem cells can be reversibly detached from bone marrow niches by applying intravenous laser therapy. In this manner, stem cells can be released rather gently and their number in the bloodstream can be substantially increased.

A first subject-matter of the present invention is therefore a method of reversibly releasing stem cells from bone marrow niches into the bloodstream of a patient in need of such treatment comprising the step of intravenously irradiating blood of the patient with laser light.

Stem cells are characterized by their ability to renew themselves and differentiate into a diverse range of specialized cell types. Hematopoietic stem cells are pluripotent (or multipotent) cells having the ability to form all blood cell types including myeloid and lymphoid lineages. In the present invention, the term “stem cell” refers to any type of stem cells. In particular, the invention deals with those stem cells that can be released from bone marrow niches into the peripheral blood stream such as hematopoietic, mesenchymal and/or endothelial stem cells. In a preferred embodiment of the invention, the stem cell is a hematopoietic stem cell.

Hematopoietic stem cells are currently used for treating certain hematological and non-hematological diseases. Mesenchymal stem cells have the potential to differentiate into various cellular lineages. Therefore, they represent a valuable source for applications in cell therapy and tissue engineering. Mesenchymal stem cells can be derived, e.g., from bone marrow. Endothelial stem cells are multipotent stem cells and can be found in bone marrow.

Intravenous irradiation of blood with laser light involves the in vivo illumination of the blood by feeding low-level laser light into a vascular channel. The part of the body into which the laser light is radiated may be chosen arbitrarily. Preferably, the laser light is radiated intravenously, e.g. into a vein of the forearm. Monochromatic laser light is inserted into the vein by means of a catheter. Intravenous laser blood irradiation was applied for the first time at the beginning of the 1980s, mainly as energy booster and health promoting source of energy and activity. The inventors of the present invention found out that intravenously applied laser light negatively charges the erythrocytes circulating in peripheral blood at the already negatively polarized glycocalix of the erythrocytes' surface by intensifying electronegativity of NANA (N-acetyl neuraminic acid)-containing membrane structures if applied intravenously.

At the same time erythrocytes are triggered into rotation by radiation with laser light at specific, defined nanometer wavelengths, which causes an electronegative field on the outer surface of the erythrocytes that has a significantly deeper and stronger electronegative effect on its immediate surrounding (also on electric fields of bio-molecules) than was already known from Van der Waals forces or the London force (more intense). During the release of stem cells from bone marrow niches the so-called “zeta potential” has a major effect on the interaction of erythrocytes that have been irradiated with laser light, are electro-negatively polarized and thus rotating with the disulphide bridges of the CXCL12-CXCR4 axis that are present as isomers in different conditions. l.v. low level laser light increases the shear plane zeta potential inside the compact layer of all erythrocytes with proton donators in form of H2 ions. These proton-donator-shear line will be able to solve those disulphide bridges between CXCL12 (also known as SDF-1 alpha), as disulphide bridges are relatively weak bindings in presence of proton donors.

Thereby, the release of stem cells from bone marrow niches becomes possible. For example, it is now possible to release at least 70% or even more of the stem cells that are positively polarized and ligated via disulphide bridges and which will be reached by the shear line of the erythrocytes.

For intravenous laser blood irradiation in terms of the present invention, low level laser light with wavelengths in the range of 350-750 nm is particularly suitable. Preferably, laser light for use in the present invention comprises green light, preferably with wavelengths in a range of 495-570 nm, in particular about 534 nm, and/or blue light, preferably with wavelengths in a range of 450-495 nm, in particular about 488 nm. In several exemplary embodiments of the invention, laser light in a range of 400-700 nm, 350-600 nm, 500-650 nm, 500-800 nm and/or 450-600 nm can be used. The power of the laser light can be, e.g., in a range of 1-3 nW, for example about 2 nW.

By applying the intravenous laser blood irradiation according to the invention, stem cells are released reversibly from bone marrow niches into the bloodstream of a patient. The patient may be a human or non-human subject. The subject may also be in need of such treatment in view of a desire to collect stem cells from the peripheral circulation of the subject for the purpose of transplantation, where the transplantation may be autologous or allogenic. In one non-limiting embodiment the subject is being prepared to donate stem cells, in particular hematopoietic stem cells.

The intravenous laser irradiation of the bloodstream is preferably carried out over a period of time that is sufficient to influence large parts of the blood of a subject with laser light. For example, the period of time is at least 30 min, preferably at least 40 min (for both wave lengths of low level laser light in case of e.g. 488 and 543 nm). This period of time is sufficient to release a substantial amount of stem cells from the bone marrow niches into the bloodstream.

In a preferred embodiment of the present invention the subject is not applied any cytokines or other factors or agents that are capable of mobilizing stem cells into circulating blood at the same time. In particular, it is preferred that the subject does not receive G-CSF, GM-CSF or CXCR4-receptor inhibitors or any derivatives, analogues, conjugates or mixtures thereof. The use of methionine will be helpful to prepare the disulphide bridges' region, as referred to in the international literature.

Furthermore, any hyaluronic acid or fragments or derivatives thereof are preferably not to be applied to the subject. If stem cells are released from bone marrow they have to pass the layer between bone marrow and bone-marrow sinusoids that contains hyaluronic acid. It was found that the additional administration of hyaluronic acid or derivatives thereof has the negative effect that this barrier effect is even increased.

In one embodiment of the present invention the release of stem cells into the bloodstream of a subject may be used to alter or modulate the relative amounts of blood cells and/or types of blood cells of the subject.

Another embodiment of the present invention further includes harvesting the mobilized stem cells from the blood stream. Thus, the present invention provides a method for providing a blood sample enriched in stem cells comprising the steps of
(i) intravenously irradiating the blood of a subject with laser light and
(ii) taking a blood sample from the subject.

The blood sample obtained by this method contains a significantly larger amount of stem cells than a blood sample from a patient whose blood has not been irradiated intravenously with laser light. Since the subject does preferably not receive any cytokines or other factors and means that are capable of mobilizing stem cells into circulating blood, the blood sample is essentially free of these substances. As far as cytokines or substances that naturally occur in a human body are concerned the definition is to be understood in that their amount is not increased as would be the case if a patient had been administered these substances externally.

Another embodiment of the present invention is a blood sample from a subject that contains an increased number of stem cells. Preferably, the number of stem cells in the blood sample is increased by at least 20-60% when compared to the number of stem cells naturally circulating in the bloodstream of a human subject. In particular, the amount of CD34+ stem cells in the peripheral blood is significantly increased.

The inventive blood sample is preferably free of factors mobilizing stem cells such as G-CSF, in particular exogenous factors mobilizing stem cells and/or factors mobilizing stem cells that have been added to the patient from which the blood sample is derived. Such factors are described herein above.

According to a preferred embodiment of the present invention, the blood sample can be used for therapeutic purposes. For this, it is possible to use the obtained blood sample directly or process the blood sample before use.

Further, it is possible to isolate stem cells from the blood sample which can then be provided for therapeutic use. Thus, another object of the present invention relates to a method of providing stem cells from a subject comprising the steps of

(i) intravenously irradiating blood of said subject with laser light,
(ii) taking a blood sample from the subject, and
(iii) isolating stem cells from said blood sample.

For isolating stem cells from the blood sample in step (iii), in principle any suitable method can be used. For example, a method, which can be used is apheresis. The cells obtained as such can then be cultivated and/or administered to a recipient subject. Usually, stem cells are collected by apheresis or another suitable method and stored as an enriched “mononucleus cell” fraction until use.

In a preferred aspect of the present invention the stem cells obtained may be used for transplantations. In principle, the stem cells can be administered into the same subject from whom they were obtained (autologous transplantation) or into a different subject (allogenic transplantation). Preferably, the administration is carried out by injection. In this way, it is possible to deliver the stem cells directly to the desired site of action. In case a systemic administration is desired, the stem cells can also be administered by infusion, thereby being transported in the blood through the body. While isolated stem cells are preferably used in transplantations, according to the invention a blood sample containing an increased number of stem cells can also be used. In this case, the blood sample preferably contains at least 20-60% more stem cells than naturally circulating in the bloodstream of a human subject.

Autologous transplants have the advantage of lower of risk of infection during the immune compromised portion of the treatment since the recovery of immune function is rapid. Also the incidents of patients experiencing rejection are very rare as the donor and recipient is the same individual.

Allogenic transplantations involve a (healthy) donor and a recipient. A disadvantage compared with autologous transplantation is that the donor must have a tissue (HLA) type that matches the recipient. But even if this is the case the administration of immune-suppressive medicaments is mandatory to mitigate graft vs. host disease.

Typical indications for transplantation, preferably autologous transplantation, are rejuvenation and tissue repair. Additional indications include lymphomas, myeloma and chronic lymphonic leukemia. For example, autologous transplantation is eligible for a patient treated with chemotherapy or radiotherapy. In this case, stem cells are reversible released in the subject using the method of the present invention and harvested from the blood stream.

The subject can then be treated with, e.g., high-dose chemotherapy and/or radiotherapy with the intention of eradicating the patient's malignant cell population at the cost of partial or complete bone marrow ablation. The patient's own stored stem cells are then transfused into his/her bloodstream where they replace destroyed tissue and resume the patient's normal blood cell production.

Of course, autologous transplantations are not limited to the above-mentioned case where a patient is treated with chemotherapy or radiotherapy. It is also possible to obtain stem cells from a subject and then apply them locally to a certain part of the body where these stem cells may be helpful. For example, transplantation of stem cells or of a blood sample enriched in stem cells can be used to provide tissue repair. Moreover, they can be used for the purpose of rejuvenation.

A further subject of the present invention is a stem cell that has been obtained by the method according to the present invention as well as a pharmaceutical composition comprising said stem cell. Preferably, the pharmaceutical composition is free of exogenous factors capable of releasing or mobilising stem cells as defined herein above.

A further advantage of the present invention is that adverse effects related to the use of factors capable of mobilizing stem cells such as anemia or reduced immunodefense/immunosuppression or autoimmune reactions that are typical, especially in the context of growth factor treatments, are avoided.

Pharmaceutical preparations may additionally comprise pharmaceutically acceptable carriers, adjuvants, excipients, stabilizing, thickening or coloring agents, binding agents, filling agents, lubricating agents, suspending agents, anti-oxidants, preservatives, etc. or components normally found in corresponding products. According to an especially preferred embodiment, the pharmaceutical preparations are in a liquid form, such as a solution for injection.

The invention is also directed to preparations comprising isolated stem cell populations derived from harvested stem cells produced by a mammalian subject by using the above methods. These preparations and/or cell populations have a clear benefit because they contain stem cells which were released reversibly from their place of origin in bone marrow niches. Therefore, these stem cells are still able to anchor in the bone marrow unless they are needed.

1. A method of reversibly releasing stem cells from bone marrow niches into the bloodstream of a subject in need of such treatment, comprising the step of intravenously irradiating blood of the subject with laser light.

2. The method of claim 1, wherein stem cells are selected from the group consisting of hematopoietic stem cells (HSC), mesenchymal stem cells (MSC), endothelial stem cells and combinations thereof.

3. The method of claim 1, wherein laser light comprises light with a wavelength in a range of 350-750 nm, in particular green light, preferably with wave lengths in a range of 495-570 nm, for example about 543 nm, and/or blue light, preferably with wave lengths in a range of 450-495 nm, for example about 488 nm.

4. The method of claim 1, wherein the subject is going to have radiation therapy or chemotherapy.

5. The method of claim 1 wherein the subject is being prepared to donate stem cells.

6. A method of providing a blood sample from a subject, wherein the blood sample comprises an increased amount of stem cells, the method comprising the steps of
(i) intravenously irradiating blood of said subject with laser light and
(ii) taking a blood sample from the subject.

7. The method of claim 6, wherein the blood sample is free of any factors capable of mobilizing stem cells into circulating blood.

8. A blood sample comprising an increased amount of stem cells, in particular hematopoietic stem cells, wherein the blood sample is free of any factors capable of mobilizing stem cells into the circulating blood.

9. A method of providing stem cells from a subject, comprising the steps of
(i) intravenously irradiating blood of said subject with laser light,
(ii) taking a blood sample from the subject and
(iii) isolating stem cells from said blood sample.

10. The method of claim 9, wherein irradiation in step (i) is carried out for at least 30 min, preferably at least 40 min.

11. A method of autologous transplantation of stem cells to a patient in need of such treatment, comprising the steps of
(i) intravenously irradiating blood of said patient with laser light,
(ii) taking a blood sample from the patient
(iii) isolating stem cells from said blood sample, and
(iv) administering the stem cells obtained in step (iii) to a desired target site of the patient.

12. Stem cell, obtained by the method of claim 9.

13. Pharmaceutical preparation, comprising a stem cell according to claim 12.
